(19) United States
(12) Patent Application Publication
(10) Pub. No.: US 2024/0327893 A1
(43) Pub. Date: Oct. 3, 2024

(54) METHOD FOR SEARCHING FOR PATHOGENIC MICROORGANISMS INSIDE THE HUMAN OR ANIMAL MICROBIOTA

(71) Applicants: Domenico Rocco BISACCIA, Pontecagnano Faiano (IT); Carlo BROGNA, Montemiletto (IT)

(72) Inventors: Domenico Rocco BISACCIA, Pontecagnano Faiano (IT); Carlo BROGNA, Montemiletto (IT)

(21) Appl. No.: 18/281,780

(22) PCT Filed: Mar. 15, 2022
(86) PCT No.: PCT/IB2022/052311

§ 371 Date: Sep. 13, 2023

(30) Foreign Application Priority Data
Mar. 23, 2021 (IT) 102021000007061

(51) Int. Cl. C12Q 1/04; C12Q 1/24; C12Q 1/689; C12Q 1/6895; G01N 30/02; G01N 30/72; G01N 30/88; G01N 33/569

(52) U.S. Cl. CPC C12Q 1/04; C12Q 1/24; C12Q 1/689; C12Q 1/6895; G01N 30/7233; G01N 30/88; G01N 33/56911; G01N 33/56961; G01N 2030/027; G01N 2030/8813; G01N 2470/04

(57) ABSTRACT

A method for searching for pathogenic microorganisms inside the human or animal microbiota, comprising the steps of collecting a human or animal biological sample wherein to search for pathogenic microorganisms, treating said biological sample by flow cytometry to individually separate the bacterial cells and the fungi present thereinside, providing a microarray with chips or wells in each of which there is in suspension a first culture medium for bacteria and a second culture medium for fungi, analysing and/or treating the material collected in said culture media.

### TECHNICAL FIELD

This invention is generally applicable to the field of microbiology and in particular it relates with a method for searching microorganisms inside the human or animal microbiota and for the possible correlation of possible pathologies with the presence of these microorganisms, such as bacteria and mushrooms, in the analyzed microbiota, also for evaluating possible therapies.

### STATE OF THE ART

With “microbiota” it is usually designated the set of all microorganisms present within a body and which defines the “microbiome”, i.e. the overall genetic heritage (genome) of these microbial populations and the interactions thereof with the host body. Several studies have shown that the microbiota inside the human body is not only the cause of disease but provides us with a very rich genome from which we take a large number of genes, necessary to survive and allow the body to adapt to continuous changes.

For this reason, the alteration of the microbiota, together with the one of the corresponding genetic heritage, can be associated with a series of side effects that can lead to one or more pathologies.

It follows that the restoration of the optimal conditions of the microbiota may represent a solution or at least an aid for the treatment of the related pathology.

### SCOPE OF THE INVENTION

The object of the present invention is to provide a technique that allows to analyze the microorganisms present in the microbiota of an organism affected by a pathology in order to correlate this pathology with the presence of these microorganisms and to evaluate the possible treatments of the pathology.

This object is achieved by the method according to the present invention, according to claim 1, which reference is made to for greater conciseness of the explanation.

Advantageous embodiments of the invention are obtained in accordance with the dependent claims.

### BEST MODES OF CARRYING OUT THE INVENTION

Some operating modes will be described here below to represent exemplary and non-limiting variants of the method according to the present invention.

These methods may be implemented within a diagnostic and/or therapeutic intervention procedure for patients suffering from pathologies whose therapy is non-surgical.

In particular, these methods may find application for patients suffering from diseases that cannot be cured by common drugs or for which it is not possible to identify the pathogenic molecules by means of common techniques, such as mass spectrometry.

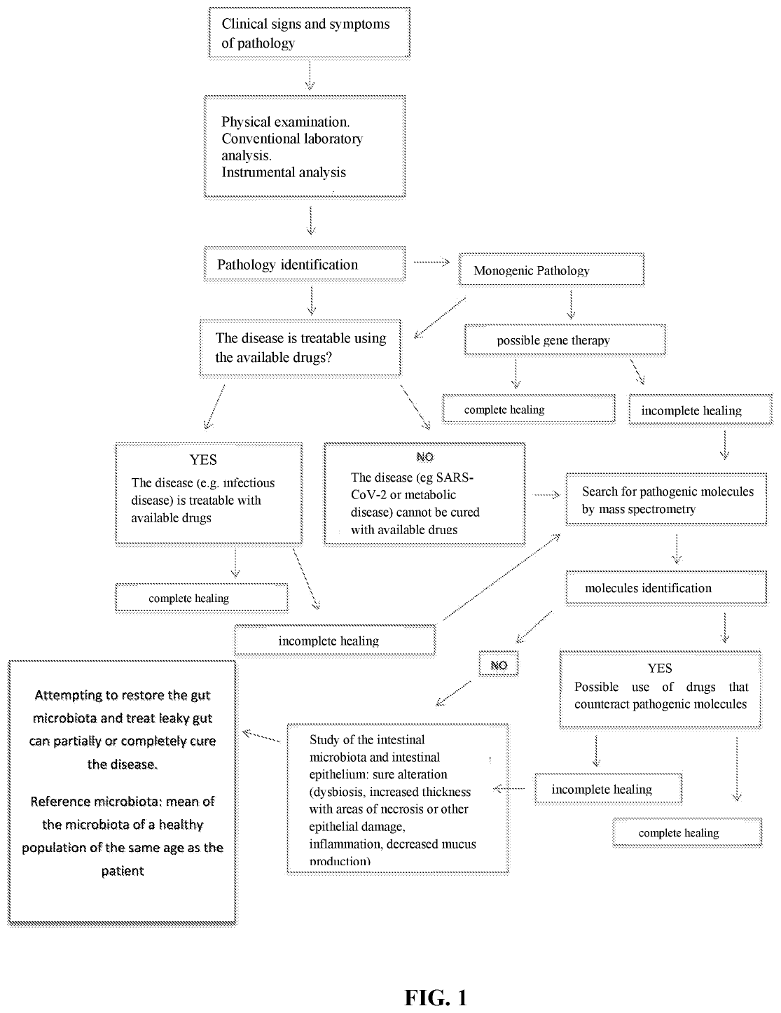

FIG. 1 shows a possible operating algorithm from which the use of analysis of the intestinal microbiota and intestinal epithelium is observed in the presence of pathologies for which the use of normal drugs or gene therapies has had no effect or has not produced complete recovery, or in those cases wherein it was not possible to identify the pathogenic molecules by means of known techniques such as mass spectrometry.

### METHOD 1

According to a first operating mode, whose object is to seek for the total microorganisms, a biological sample is first of all collected from the patient suffering from a pathology for which a possible involvement of the intestinal microbiota is suspected. Specifically, the biological sample will be a fecal sample.

The sample is subjected to single cell flow cytometry by means of a flow cytometer with volumetric counting using an aqueous solution of water/PES as eluent.

In this way it is possible to separate the bacterial cells and fungi individually, which will then be inserted individually in the chips of a suitably prepared microarray, with a flow rate per cell sample that will be defined by the type of flow cytometer.

Two media are present in suspension in each of these chips: a first non-selective culture medium for bacteria, such as McConkey agar, and one for fungi, such as Sabouraud dextrose agar.

These media must not be fluorescent and must favor bacterial or fungal activity.

At this point, depending on the material obtained, four different methods may be carried out for each chip.

A first method involves cell culture on selective media.

A second possible method provides the use of RT-PCR (Real Time, Polymerase Chain Reaction) on the genetic material of individual bacteria/fungi to examine the genome and consequently the species. There may be small differences between bacteria/fungi belonging to a single species and this could have a significant value for the patient's pathology, therefore it is necessary to “design” specific and less specific primers.

A third possibility involves the analysis of the viral load present in the chips, for example by means of a PCR or antigen test buffer, as bacteria/fungi may be carriers of viruses, such as the SARS-CoV-2 coronavirus.

Finally, a fourth operative methodology provides the filtration of the suspension present in each chip and inoculation in experimental animals (for example mice or rabbits) or in the blood or in organoids to evaluate the reaction of the tissue or of the immune system.

### METHOD 2

A second operating mode, whose object is to seek for molecule-producing microorganisms, provides the use of mass spectrometry to identify the molecules in the patient's serum (e.g. toxins).

Also in this case, a biological sample is first of all acquired, preferably a fecal sample, in order to recover many bacteria and fungi, including those producing the molecules found in the serum.

It goes on in a similar way to the first operating mode, separating the bacterial cells and fungi individually by flow cytometry by means of flow cytometry provided with volumetric counting using aqueous solution of water/PES as eluent fluid, and then individually introducing the cells into the microarray chips.

The eluent fluid must not affect the properties and/or the vitality of the bacteria and fungi.

In each of these chips there are two media in suspension: a first non-selective culture medium for bacteria, for example McConkey agar, and one for fungi, such as Sabouraud dextrose agar, which have the property of not being fluorescent and of favoring the bacterial or fungal activity.

Chips containing the same bacteria or fungi may be present, depending on the proportion in which they are present in the fecal sample.

In addition to the previous method, the chips also contain antibodies and/or fluorescence enzymes attached to the bottom of the chips and which recognize the molecules found previously.

When contact with these molecules occurs, and only with them, in the case of antibodies, identification will be carried out with the sandwich ELISA technique, with which the antigen or molecule will be captured, while in the case of fluorescence enzymes the evidence of the presence of the molecule will be provided by the fact that these enzymes change fluorescence when they bind the molecule.

The use of antibodies has the advantage of having high specificity but, on the other hand, has high costs, requires an additional waiting time to obtain the antibody and therefore also a further step in the procedure.

The use of enzymes has good specificity, even if lower than the previous case, and low costs, but requires a high number of enzymes with specific receptors.

In both cases it will be possible to know the bacterium or fungus responsible for the production of these molecules, regardless of whether or not conditioned by a phage, to promote its growth in order to identify it and extract its DNA for analysis.

The phage cycle must be lysogenic and not lytic.

It should be noted that if the molecules sought in the serum are enzymes, they will be identified with a fluorescent molecule that acts as a substrate, changing the fluorescence once the contact has taken place.

### METHOD 3

A third operating mode, whose object is to seek for molecule-producing microorganisms, provides the starting use of mass spectrometry to identify the molecules in the patient's serum (e.g. toxins).

Also in this case, a biological sample is first of all acquired, preferably a fecal sample, in order to recover many bacteria and fungi, including those producing the molecules found in the serum.

It goes on in a similar way to the previous operating modes, separating the bacterial cells and fungi individually by flow cytometry by means of flow cytometry provided with volumetric counting using aqueous solution of water/PES as eluent fluid, and then individually introducing the cells into the chips of the microarray.

The eluent fluid must not affect the properties and/or the vitality of the bacteria and fungi.

In each of these chips there are two media in suspension: a first non-selective culture medium for bacteria, for example McConkey agar, and one for fungi, such as Sabouraud dextrose agar, which have the property of not being fluorescent and of favoring the bacterial or fungal activity.

Growing bacteria and/or fungi will produce the sought molecules.

The solution is filtered with ultrafilters and analyzed with UHPLC-HRMS (Ultra High Performance Liquid Chromatography+High Resolution Mass Spectrometry).

This solution has the advantage of having very high specificity even if limited by high cost.

This analysis allows to know which bacterium or fungus is responsible for the production of these molecules, regardless of whether or not it is conditioned by a phage, to promote its growth and identify it, so as to extract its DNA for subsequent analysis.

The phage cycle must be lysogenic and not lytic.

### METHOD 4

A fourth operating mode, whose object is to seek for molecule-producing microorganisms, provides the initial use of mass spectrometry to identify the molecules in the patient's serum (e.g. toxins).

A biological sample is then acquired, preferably a fecal sample, in order to recover many bacteria and fungi, including those producing the molecules found in the serum.

The sample is incubated with one or more viruses, for example viruses naturally present in the human body, which behave like phages, labeled with the 32P isotope. The sample is then washed and/or filtered to remove the viruses.

The procedure continues, similarly to the previous operating methods, separating the bacterial cells and fungi individually by flow cytometry by means of flow cytometry provided with volumetric counting using aqueous solution of water/PES as eluent fluid, and then individually introducing the cells into the microarray chips. The eluent fluid must not affect properties and/or the vitality of the bacteria and fungi.

In each of these chips there are two media in suspension: a first non-selective culture medium for bacteria, for example McConkey agar, and one for fungi, such as Sabouraud dextrose agar, which have the property of not being fluorescent and of favoring the bacterial or fungal activity.

Only the bacteria and fungi that will make the respective chip fluorescent will be cultured as they are the ones that produce the molecules sought, as they are the bacteria and/or fungi affected by the phage.

In this way it will be possible to know which bacterium/fungus is responsible for the production of the molecules, regardless of whether or not it is conditioned by a phage, to favor its growth for the purpose of its identification and subsequent DNA extraction for analysis purposes.

This procedure has the advantage of presenting high specificity but its drawback is the need for virus manipulation.

Also in this case, the phage cycle must be lysogenic and not lytic.

1. A method for searching for pathogenic microorganisms inside the human or animal microbiota, comprising the following steps:
a) collecting a human or animal biological sample wherein to search for pathogenic microorganisms;
b) treating said biological sample by flow cytometry to individually separate the bacterial cells and the fungi present thereinside;
c) providing a microarray with chips or wells in each of which there is in suspension a first culture medium for bacteria and a second culture medium for fungi different from said first culture medium, for introducing respectively said bacterial cells and said fungi separated in said step b);
d) analysing and/or treating the material collected in said culture media.

2. Method as claimed in claim 1, characterized in that said step d) is selected from one of the following steps:
cell culture on selective media;
carrying out RT-PCR (Real Time, Polymerase Chain Reaction) on single bacteria/fungi to examine their genome and consequently the species;
analysis of the viral load present in said chips, for example by means of a PCR or antigen test buffer;
filtrating the suspension present in each of said chips and inoculating in experimental animals or in the blood or in organoids to evaluate the reaction of the tissue or of the immune system.

3. Method as claimed in claim 1, characterized by providing, upstream of said step a) of collecting said biological sample, a step e) of carrying out a mass spectrometry to identify the molecules present in the patient's serum.

4. Method as claimed in claim 2, wherein said chips also contain antibodies and/or fluorescent enzymes bound to the bottom and which recognize the molecules found in said step e).

5. Method as claimed in claim 3, wherein said step d) provides for the recognition of the bacterium or fungus responsible for the production of said molecules by means of the sandwich ELISA technique, if antibodies are used, or use of fluorescent receptors, if enzymes are used.

6. Method as claimed in claim 2, wherein said step d) provides for the filtering of the solution with ultrafilters and subsequent analysis with UHPLC-HRMS (Ultra High Performance Liquid Chromatography+High Resolution Mass Spectrometry) for the determination of the bacterium or fungus responsible for the production of said molecules.

7. Method as claimed in claim 6, wherein a growth and identification step of said bacterium or fungus is carried out for the subsequent extraction and analysis of its DNA.

8. Method as claimed in claim 3, wherein, upstream of said step b), a step is provided for the incubation of said sample with one or more viruses having phage function, labeled with isotope 32P and for the subsequent removal of said one or more viruses.

9. Method as claimed in claim 8, wherein only the bacteria and/or fungi affected by the phage and which will make the respective chip fluorescent will be cultured to determine which bacterium or fungus is responsible for the production of said molecules, a subsequent step being also provided for the growing and the identification of said bacterium or fungus for the subsequent extraction and analysis of its DNA.

10. Method as claimed in claim 1, wherein said biological sample is a fecal sample.
